(19) United States
(12) Patent Application Publication
(10) Pub. No.: US 2009/0221502 A1
(43) Pub. Date: Sep. 3, 2009

(54) Vegetarian anabolic protein composition

(75) Inventors: Michael Todd Yatcilla, Los Angeles, CA (US); Geoffrey Michael Palmer, Pompano Beach, FL (US)

Correspondence Address: GABOR L. SZEKERES, P.O. BOX 27938, ANAHEIM HILLS, CA, 92809, US

(73) Assignee: Natrol, Inc.

(21) Appl. No.: 12/380,448

(22) Filed: Feb. 27, 2009

Related U.S. Application Data
(60) Provisional application No. 61/067,871, filed on Mar. 3, 2008.

(51) Int. Cl. A61K 38/16

(52) U.S. Cl. 514/12

(57) ABSTRACT

Vegetarian amino acid sources are combined to create a protein blend that closely matches human muscle tissue in terms of amino acid composition. “Closely matching” means that the amino acid composition of the protein blend is within five percent (5%) of the amino acid composition of human muscle for all of the amino acids of the human muscle. The protein blend can be used as food or food supplement to facilitate and bolster muscle anabolism for healthy humans desiring such result and also to treat and or ameliorate the effects of certain diseases and conditions such as sarcopenia, cancer cachexia, HIV wasting, malnutrition, primary muscle diseases (myopathy) and side effects of corticosteroid therapy.

### CLAIM OF PRIORITY OF PROVISIONAL APPLICATION

The present application claims the priority of U.S. provisional application 61/009,107 filed on Dec. 24, 2007.

### BACKGROUND OF THE INVENTION

1. Field of the Invention

The invention relates to nutritional supplements and specifically to an orally ingested completely vegetarian protein supplement that closely matches the amino acid composition of human muscle and improves physical performance and overall health and is also useful for treating or ameliorating certain human diseases and conditions.

2. Brief Description of the Prior Art

High-intensity weight (resistance) training causes muscle anabolism by multiple mechanisms. (Godfrey R J. et al. Sports Medicine 2003; 33(8): 599-613; Falk et al. Pediatr Endocrinol Rev 2003; 1(2): 120-7). Although the inventors do not wish to be bound by theory, a prominent theoretical basis for rate-limitation in muscle anabolism is the “depletion theory.” According to this theory, the extent and rate of muscle anabolism is limited by whichever micro- or macro-nutrient is first depleted as “building blocks” in the nutrient supply to the growing muscle tissue. Hence weight trainers and other athletes who seek muscle hypertrophy commonly take protein dietary supplements. The common protein dietary supplements recommended and consumed to attain muscle anabolism include concentrated or isolated whey or soy proteins as their main component. The whey and soy proteins are usually obtained as byproducts of cheese or of soybean oil manufacture, respectively, and they are indeed reasonably good quality dietary proteins that show good benefit in human nutrition. (Yalcin. Curr Phar. Des. 2006; 12(13): 1637-43; Ha et al. J Nutr Biochem. 2003; 14(5):251-8; Haub et al. Am J Clin Nutr. 2002; 76(3): 511-7; Jeejeebhoy. Eur J Gastroenterol Hepatol. 2000; 12(1): 1-2.

However, for the specific purpose of muscle anabolism, it would be desirable and would attain better results to have a dietary supplement of a composition that, as far as amino acids are concerned, most closely matches the composition of human muscle. As Table 1 below shows whey and soy proteins do not closely match the amino acid composition of human muscle because they are deficient in several amino acids that are present in human muscle tissue. In accordance with the depletion theory of anabolism and practical considerations also, the lack or mismatch in percentage of certain amino acids between the respective protein supplement and human muscle is likely to limit the rate at which human muscle anabolism could be obtained.

TABLE 1
Human muscle composition (Matthews. Modern Nutrition in
Health and Disease. Baltimore, Md: Williams & Wilkins; 2006:
23-58) along with soy (Karr-Lilienthal et al. J. Agric Food Chem.
2004; 52(20): 6193-9) and whey protein isolates (McDonough
et al. J Dairy Sci 1974; 57(12): 1438-43)

| | Human Muscle | Percentage | Soy | Whey Protein |
|---|---|---|---|---|
| 3-Letter Amino | Composition | in human | Protein | Isolate |
| Acid Code | (mg/g) | muscle | (mg/g) | (mg/g) |
| ILE | 31.5 | 5.18 | 43 | 51 |
| LEU | 53.3 | 8.76 | 71 | 113 |
| LYS | 56.6 | 9.3 | 55 | 104 |
| MET | 16.9 | 2.78 | 11 | 21 |
| PHE | 29.9 | 4.91 | 45 | 33 |
| THR | 31 | 5.1 | 33 | 42 |
| TRP | 7.5 | 1.23 | 12 | 25 |
| VAL | 36.7 | 6.03 | 44 | 48 |
| CYS | 9.7 | 1.59 | 11 | 15 |
| HIS | 18.6 | 3.06 | 23 | 20 |
| TYR | 20.5 | 3.37 | 33 | 34 |
| ALA | 43.5 | 7.15 | 37 | 45 |
| ARG | 44.2 | 7.26 | 66 | 22 |
| GLX | 54.8 | 9.01 | 166 | 156 |
| GLY | 33.6 | 5.52 | 37 | 15 |
| PRO | 30.2 | 4.96 | 44 | 41 |
| SER | 36.9 | 6.07 | 0 | 26 |
| ASX | 53 | 8.71 | 101 | 105 |
| Total mg/g | 608.4 | 99.99 | 832 | 916 |

An additional problem in the current art is that the number of vegetarian and vegan consumers is increasing while most high quality protein sources are derived from animal ingredients, specifically whey proteins, milk proteins, and egg proteins. Vegetarian protein sources are highly deficient in multiple amino acids (see soy in Table 1). Moreover, dependence on a single source of protein supply, such as whey or soy protein isolate or concentrate, is considered undesirable in the art for multiple reasons, including commercial ones.

There is therefore an unmet need in the art to supply a dietary protein supplement that closely matches the amino acid composition of human muscle tissue, is not deficient in any amino acids, is completely vegetarian and does not depend on a single protein source. The dietary supplement composition of the present invention meets this need.

### SUMMARY OF THE INVENTION

Vegetarian amino acid sources are combined to create a protein blend that closely matches human muscle tissue in terms of amino acid composition. In the context of this application “closely matching” means that the amino acid composition of the protein blend is within five percent (5%) of the amino acid composition of human muscle for all of the amino acids, as shown in Table 1. The protein blend can be used as food or food supplement to facilitate and bolster muscle anabolism for healthy humans desiring such result and also to treat and or ameliorate the effects of certain diseases and conditions such as sarcopenia, cancer cachexia, HIV wasting, malnutrition, primary muscle diseases (myopathy) and side effects of corticosteroid therapy.

### DETAILED DESCRIPTION OF THE INVENTION

Soy protein isolate, pea protein concentrate, rice protein isolate, l-lysine, l-glycine and l-alanine are mixed to match the human muscle composition. The amino acid composition of the blends is compared to the amino acid composition of human muscle. A “match” of each combination of the five (5) ingredients is assessed by computing the total sum of squares of differences between the amino acid compositions of the blend with that of human muscle.

Best fit to human muscle is established by minimizing the total sum of squares of differences between the blend and human muscle by varying the five (5) ingredients using any automated algorithm. Any algorithm that utilizes the method of least squares (see for example Å. Björck, Numerical Methods for Least Squares Problems, Cambridge, 1996) can be used; a suitable one is the SOLVER plug-in (Frontline Systems, Inc., Incline Village, Nev.) to Microsoft Excel (Microsoft Corporation, Redmond, Wash.). An additional constraint is added that the resultant blend may not be lacking any amino acid of the human muscle and must be a “close match” as defined above. Preferably no amino acid is present in the blend in lesser percentage than in the human muscle as shown in Table 1.

In accordance with the following considerations a dietary composition is provided in accordance with the present invention that, in addition to usual and/or optional excipients, contains the following combination of ingredients:

Two or more vegetable proteins selected from the group consisting of soy protein, rice protein, pea protein, buckwheat protein, wheat protein, potato protein, sunflower protein, safflower protein, hops protein and mustard protein, and one or more of the isolated amino acids selected from the group consisting of lysine, glycine and alanine, with the further condition that the amino acid composition of the blend closely matches the amino acid composition of the human muscle, as disclosed in Table 1. Acceptable percentages of ingredients the appropriate combination of which can provide the blend satisfying the above stated criteria are, for example
1.0-8.5% soy protein isolate
10.5-19% pea protein isolate
72-76% rice protein isolate
1.5-3.5% lysine and
0.5-1.5% glycine
and
1-20% buckwheat
24-32% pea
46-62% rice
1.5-3.5% lysine
0.5-1.5% glycine
1.5-2.5% alanine

Applicants understand that the total percentage of the active ingredients or components shown above (and not even considering optional excipients) cannot exceed 100%. Therefore, the actual percentages of the active ingredients are to be selected such that their total is 100%, and this 100% of the active components is then diluted with optional excipients well known in the art.

The dietary composition or blend of the present invention may be in the form of tablets, capsules, two-piece hard gelatin capsules, effervescent tablets, powdered drink mix, drink blend and protein bar. The foregoing indicated percentages refer only to the listed ingredients and do not include the weight of the other excipeints that are usually present in the composition or formulation.
Tablets are the presently one preferred composition of the blend of the invention. A broad example of the composition of the tablet or other unitary dose of the composition is shown below.

| | Amount |
|---|---|
| Ingredient | Per Tablet |
| Soy Protein Isolate | 10-900 |
| Pea Protein Isolate | 10-900 |
| Rice Protein Isolate | 10-900 |
| L-Lysine HCl Gran (80% Lysine) | 1-100 |
| Glycine USP (Aminoacetic Acid) | 1-100 |

The numbers refer to milligrams (mg) of ingredient per single tablet, capsule or other unitary dose of the formulation. In addition to the listed ingredients usual pharmaceutically acceptable excipients may also be present in the tablet or other unitary dose.

More preferably, in addition to usual and/or optional excipients, the formulation contains the following combination of ingredients in the amounts indicated. Again, the numbers refer to milligrams of ingredient per single tablet, capsule or other unitary dose of the formulation.

| | Amount |
|---|---|
| Ingredient | Per Tablet |
| Soy Protein Isolate | 20-500 |
| Pea Protein Isolate | 20-500 |
| Rice Protein Isolate | 400-900 |
| L-Lysine HCl Gran (80% Lysine) | 5-75 |
| Glycine USP (Aminoacetic Acid) | 5-75 |

Even more preferably, in addition to usual and/or optional excipients, the formulation contains the following combination of ingredients in the amounts indicated. (The numbers refer to milligrams of ingredient per single tablet, capsule or other unitary dose of the formulation.)

| | Amount |
|---|---|
| Ingredient | Per Tablet |
| Soy Protein Isolate | 50-100 |
| Pea Protein Isolate | 50-150 |
| Rice Protein Isolate | 600-900 |
| L-Lysine HCl Gran (80% Lysine) | 10-50 |
| Glycine USP (Aminoacetic Acid) | 5-20 |

In terms of percentages of the essential ingredients (not considering the weight of excipients), examples of the formulation are

### (1) a Protein Composition Essentially Consisting of

8% soy protein isolate
11% pea protein isolate
74% rice protein isolate
3% l-lysine
1% glycine,
and

### (2) a Protein Composition Essentially Consisting of

19% buckwheat protein concentrate (available from Cyvex Nutrition, Irvine, Calif.),
28% pea protein isolate 46%,
rice protein isolate 2%,
l-lysine 1%,
glycine 2%,
beta-alanine (available from Natural Alternatives International, San Marcos, Calif.).
   The weights in milligrams or percentages in the exemplary formulations above are selected in such a way that the criteria of closely matching the amino acid composition of the human muscle are met.
   Another preferred exemplary formulation of the anabolic protein composition of the invention is in the form of a powder that can be sweetened and flavored to be mixed with water and taken as a beverage. A unitary dose, recommended as a single daily dose, of this exemplary formulation has the following ingredients in the following ranges.
Soy protein isolate 0.1 to 30 grams;
Pea protein isolate 0.1 to 30 grams;
Rice protein isolate 0.1 to 30 grams;
L-Lysine HCl Gran (80% Lysine) 0.1 to 3 grams;
Glycine USP (aminoacetic acid) 0.1 to 3 grams;
Maltodextrin (optional) 1 to 10 grams;
Sucralose (optional sweetener, or other sweetener) 0.05 to 0.5 grams;
Acesulfame potassioum (optional) 0.001 to 0.02 grams, and
Natural and/or artificial vanilla flavor (optional., or other flavor) 0.1 to 1 gram.
   This unitary dose is designed to be mixed with approximately 8 ounces of cold water and consumed as a beverage.

### Presently Preferred Actual Formulation, Specific Embodiment

The presently preferred specific embodiment has the following five (5) essential ingredients of the inventive composition as shown below. (The numbers refer to milligrams per tablet, or other formulation such as a capsule or the like.)

| | Amount |
|---|---|
| Ingredient | Per Tablet |
| Soy Protein Isolate | 80 |
| Pea Protein Isolate | 110 |
| Rice Protein Isolate | 740 |
| L-Lysine HCl Gran (80% Lysine) | 37.5 |
| Glycine USP (Aminoacetic Acid) | 10.15 |

Table 2 below shows in a different manner the best-fit combination of the five (5) essential ingredients, soy protein isolate, pea protein concentrate, rice protein isolate, l-lysine and l-glycine that is all vegetarian and best approximates in terms of amino acid composition the composition of human muscle. This best fit combination is the presently most preferred and specific embodiment of the present invention. As is indicated in the table weights, ratios or proportions are indicated as milligram per gram (mg/g). Table 2 also provides comparison with whey protein isolate that is not used in accordance with the present invention.

TABLE 2
Amino Acid composition of the preferred embodiment

| | | | | | | | | | I | | K |
|---|---|---|---|---|---|---|---|---|---|---|---|
| | | B | | | | | | | Square of | J | Square of |
| | A | Human | C | D | E | | | H | Difference | Whey | Difference |
| | 3-Letter | Muscle | Soy | Pea | Rice | F | G | Invented | (invention − | Protein | (whey − |
| | Amino | Comp | Protein | Protein | Protein | Lysine | Glycine | composition | human | Isolate | human |
| 1 | Acid Code | (mg/g) | (mg/g) | (mg/g) | (mg/g) | (mg) | (mg) | (mg/g) | muscle) | (mg/g) | muscle) |
| 2 | ILE | 31.5 | 43 | 43.9 | 39.6 | | | 37.6 | 37 | 51 | 380 |
| 3 | LEU | 53.3 | 71 | 85.1 | 74.8 | | | 70.4 | 292 | 113 | 3564 |
| 4 | LYS | 56.6 | 55 | 76.9 | 30.6 | 800 | | 59.5 | 8 | 104 | 2247 |
| 5 | MET | 16.9 | 11 | 7.2 | 28.5 | | | 22.8 | 34 | 21 | 17 |
| 6 | PHE | 29.9 | 45 | 57.6 | 49 | | | 46.2 | 266 | 33 | 10 |
| 7 | THR | 31 | 33 | 120 | 33.12 | | | 40.3 | 87 | 42 | 121 |
| 8 | TRP | 7.5 | 12 | 7.9 | 10.8 | | | 9.8 | 5 | 25 | 306 |
| 9 | VAL | 36.7 | 44 | 49.3 | 54 | | | 48.9 | 149 | 48 | 128 |
| 10 | CYS | 9.7 | 11 | 8.3 | 22 | | | 18.1 | 70 | 15 | 28 |
| 11 | HIS | 18.6 | 23 | 24.5 | 19 | | | 18.6 | 0 | 20 | 2 |
| 12 | TYR | 20.5 | 33 | 39.6 | 48.6 | | | 43 | 504 | 34 | 182 |
| 13 | ALA | 43.5 | 37 | 40 | 48.9 | | | 43.5 | 0 | 45 | 2 |
| 14 | ARG | 44.2 | 66 | 8.2 | 82.3 | | | 67.1 | 524 | 22 | 493 |
| 15 | GLX | 54.8 | 166 | 184.5 | 154.9 | | | 148.2 | 8724 | 156 | 10241 |
| 16 | GLY | 33.6 | 37 | | 38.2 | | 985 | 41.1 | 56 | 15 | 346 |
| 17 | PRO | 30.2 | 44 | 40.8 | 44.8 | | | 41.2 | 120 | 41 | 117 |
| 18 | SER | 36.9 | | 53.2 | 43.2 | | | 37.8 | 1 | 26 | 119 |
| 19 | ASX | 53 | 101 | 120 | 77.5 | | | 78.6 | 657 | 105 | 2704 |
| 20 | Total mg/g | 608.4 | 832 | 967 | 899.82 | | | 838.8 | | 916 | |
| 21 | Cost/kg | | $5.70 | $6.50 | $6.75 | $6.83 | $6.32 | $6.43 | | $9.35 | |
| 22 | Fraction | | 0.08 | 0.11 | 0.74 | 0.03 | 0.01 | | | | |
| 23 | Sum of | | | | | | | | 11534.9 | | 21007 |
| | Square of | | | | | | | | | | |
| | Differences | | | | | | | | | | |

By way of explanation of Table 2, Column B shows the amino acid composition of human muscle tissue as milligrams of amino acid per gram of human muscle tissue. (Matthews. Modern Nutrition in Health and Disease. Baltimore, Md.: Williams & Wilkins; 2006:23-58).

Columns C, D, and E show the typical amino acid composition of soy protein isolate obtained from a typical commercial source (for example from the Archer Daniels Midland Corporation, Decatur, Ill.), of pea protein isolate obtained from a typical commercial source (Parrheim Foods, Saskatoon, SK, Canada), and rice protein isolate obtained from a typical commercial source (Oryzatein™, Axiom Foods, Santa Monica, Calif.) as milligrams of amino acid per gram of total product.

Columns F and G show the “amino acid composition” of the pure amino acid materials l-lysine obtained from a typical commercial source (Ajinomoto Amino Science LLC, Raleigh, N.C.) and of glycine obtained from a typical commercial source (Ajinomoto Amino Science LLC, Raleigh, N.C.).

Column H shows the amino acid composition of the combination of soy protein, pea protein, rice protein, lysine and glycine in the ratios prescribed by row 22 (see below). This is the amino acid composition of the preferred embodiment of the presently invention.

Column I shows the Square of the Differences between the “Invention” amino acid composition (column H) and the amino acid composition of human muscle tissue (column B). These numbers are added together to give the total sum of the square of the differences, which is reported in row 23.

Column J shows the amino acid composition of whey protein isolate obtained from a typical commercial source (Davisco Foods International, Inc. Le Sueur, Minn.) by comparison.

Column K shows the Square of the Differences between whey protein amino acid composition (column J) and the amino acid composition of human muscle tissue (column B). These numbers are added together to give the total sum of the square of the differences, which is reported in row 23.

Row 21 shows the current commercial cost per kilo for the various protein and amino acid sources.

Row 22 shows the relative fraction of the five ingredients (soy protein, pea protein, rice protein, lysine, and glycine) combined to make the presently preferred specific embodiment of the invention. The relative fractions sum to nearly one (1) disclosing the proportions of all essential ingredients of the composition.

The vegetarian blend shown in Table 2 exhibits a fit to human muscle protein better than any of the pure protein sources (soy, whey, rice, pea) and is superior to any other combination of ingredients. It additionally is less expensive than whey protein and can be considered 100% “vegetarian.”

This blend was combined along with other vegetarian excipient ingredients such as microcrystalline cellulose, calcium phosphate dibasic, croscarmellose sodium, stearic acid, silicon dioxide, magnesium stearate and a hydroxypropylmethylcellulose/triacetin coating into a formulation shown in Table 3. Although a tablet formulation is the presently preferred specific embodiment of the invention, those skilled in the art will readily understand that the protein and amino acid components of the invention can be combined in the ratios indicated above with additional and/or other excipients to provide other tablet and other well known formulations, such as without limitation capsules, powders for drinks, protein bars, that are within the scope of the invention.

TABLE 3
Tablet formulation of one specific embodiment

| | Amount |
|---|---|
| Ingredient | Per Tablet |
| Soy Protein Isolate | 80 |
| Pea Protein Isolate | 110 |
| Rice Protein Isolate | 740 |
| L-Lysine HCl Gran (80% Lysine) | 37.5 |
| Glycine USP (Aminoacetic Acid) | 10.152 |
| Microcrystalline Cellulose | 200 |
| Calcium Phosphate Dibasic | 200 |
| Croscarmellose Sodium | 50 |
| Stearic acid | 60 |
| Silicon dioxide | 20 |
| Magnesium Stearate | 20 |
| Aqueous film coating consisting | 15 |
| Of hydroxypropylmethylcellulose | |
| And triacetin (Opadry YS-2-7035) | |

The formulation shown in Table 3 was blended using a cross-flow powder mixer, compressed into tablets using a rotary tablet press of standard design and coated using an aqueous film coater of standard design.

A specific embodiment of the formulation that is to be used as a powder mixed with water and consumed as a beverage is as follows.
Soy protein isolate 2.4 grams;
Pea protein isolate 3.3 grams;
Rice protein isolate 22.2 grams;
L-Lysine HCl Gran (80% Lysine) 1.1 grams;
Glycine USP (aminoacetic acid) 0.3 grams;
Maltodextrin 2.9 grams;
Sucralose 0.19 grams;
Acesulfame potassioum 0.006 grams, and
Natural & artificial vanilla flavor 0.48 grams.
   This unitary dose is to be mixed with approximately 8 ounces of cold water.

The vegetarian tablets or LIKE formulations in accordance with the invention are to be swallowed by humans to induce or increase muscle anabolism in healthy humans who desire such result and for treating and or ameliorating the effects of certain diseases and conditions such as sarcopenia, cancer cachexia, HIV wasting, malnutrition, primary muscle diseases (myopathy) and side effects of corticosteroid therapy.

The recommended dosage is 1 to 20 tablets (or equivalent unitary dose formulations) per day, more preferably 5 to 15 tablets (or equivalent unitary dose formulations) per day, and most preferably nine (9) tablets (or equivalent unitary dose formulations) per day. In case of the formulation to be used as a powder to be mixed with water and consumed as a beverage the recommended dose is one unitary dose, as defined above, per day.

What is claimed is:

1. A composition for affecting anabolism in a human comprising the combination of
a) a plurality of vegetable proteins selected from the group consisting of soy protein, rice protein, pea protein, buckwheat protein, wheat protein, potato protein, sunflower protein, safflower protein, hops protein and mustard protein and
b) one or more isolated amino acids selected from the group consisting of lysine, glycine and alanine,
the composition closely matching with a difference of no more than five percent (5%) for each amino acid, the amino acid composition of human muscle.

2. A composition in accordance with claim 1 where the amino acid composition of the human muscle is as follows in terms of weight percentage relative to the total amino acid composition of the muscle shown below, and wherein no amino acid of the composition is less than the corresponding percentage in the human muscle.

| Amino Acid | Percentage in |
|---|---|
| 3 letter code | human muscle |
| ILE | 5.18 |
| LEU | 8.76 |
| LYS | 9.3 |
| MET | 2.78 |
| PHE | 4.91 |
| THR | 5.1 |
| TRP | 1.23 |
| VAL | 6.03 |
| CYS | 1.59 |
| HIS | 3.06 |
| TYR | 3.37 |
| ALA | 7.15 |
| ARG | 7.26 |
| GLX | 9.01 |
| GLY | 5.52 |
| PRO | 4.96 |
| SER | 6.07 |
| ASX | 8.71 |

3. A composition in accordance with claim 1 comprising:
soy protein isolate, pea protein concentrate, rice protein isolate, l-lysine and l-glycine.

4. A composition in accordance with claim 2 comprising:
soy protein isolate, pea protein concentrate, rice protein isolate, l-lysine and l-glycine.

5. A composition in accordance with claim 1 where in one unitary dose of the supplement the ingredients are present in the following amounts:
10 to 900 mg soy protein isolate per unitary dose;
10 to 900 mg pea protein isolate per unitary dose;
10 to 900 mg rice protein isolate per unitary dose
L-lysine HCl Gran (containing 80% L-lysine) 1 to 100 mg per unitary dose, and
glycine USP 1 to 100 mg per unitary dose.

6. A composition in accordance with 5 where in one unitary dose of the supplement the ingredients are present in the following amounts:
20 to 500 mg soy protein isolate per unitary dose;
20 to 500 mg pea protein isolate per unitary dose;
400 to 900 mg rice protein isolate per unitary dose
L-lysine HCl Gran (containing 80% L-lysine) 5 to 75 mg per unitary dose, and
glycine USP 5 to 75 mg per unitary dose.

7. A composition in accordance with claim 6 where in one unitary dose of the supplement the ingredients are present in the following amounts:
50 to 100 mg soy protein isolate per unitary dose;
50 to 150 mg pea protein isolate per unitary dose;
600 to 900 mg rice protein isolate per unitary dose
L-lysine HCl Gran (containing 80% L-lysine) 10 to 50 mg per unitary dose, and
glycine USP 5 to 25 mg per unitary dose.

8. A composition in accordance with claims 1 additionally comprising a pharmaceutically acceptable excipient.

9. A composition in accordance with claims 2 additionally comprising a pharmaceutically acceptable excipient.

10. A powder composition to be used mixed with water as a beverage, for inducing muscle anabolism in a human, one unitary dose of the composition comprising the combination of
Soy protein isolate 0.1 to 30 grams;
Pea protein isolate 0.1 to 30 grams;
Rice protein isolate 0.1 to 30 grams;
L-Lysine HCI Gran (80% Lysine) 0.1 to 3 grams;
Glycine USP (aminoacetic acid) 0.1 to 3 grams;
the composition closely matching with a difference of no more than five percent (5%) for each amino acid, the amino acid composition of human muscle.

11. A powder composition in accordance with claim 10, wherein one daily unitary dose comprises a combination of
Soy protein isolate approximately 2.4 grams;
Pea protein isolate approximately 3.3 grams;
Rice protein isolate approximately 22.2 grams;
L-Lysine HCl Gran (80% Lysine) approximately 1.1 grams;
Glycine USP (aminoacetic acid) approximately 0.3 grams;
Maltodextrin approximately 2.9 grams;
Sucralose approximately 0.19 grams;
Acesulfame potassioum approximately 0.006 grams, and
Natural and/or artificial vanilla flavor approximately 0.48 grams.

12. A method of increasing muscle anabolism for a human, the method comprising the step of administering to a human composition as defined in claim 1.

13. A method in accordance with claim 12 wherein the composition is administered to increase muscle anabolism for healthy humans desiring such result or to treat and or ameliorate the effects of certain diseases and conditions selected from the group consisting of sarcopenia, cancer cachexia, HIV wasting, malnutrition, primary muscle diseases (myopathy) and side effects of corticosteroid therapy.

14. A method in accordance with claim 13 wherein the composition is as defined in claim 2.

15. A method in accordance with claim 13 wherein the composition is as defined in claim 5.

16. A method in accordance with claim 13 wherein the composition is as defined in claim 6.

17. A method in accordance with claim 13 wherein the composition is as defined in claim 7.

18. A method in accordance with claim 13 wherein the composition is as defined in claim 10.

19. A method in accordance with claim 13 wherein the composition is as defined in claim 11.

20. 18. A method in accordance with claim 13 wherein the composition is administered to increase muscle anabolism for healthy humans desiring such result.
